(19) United States
(12) Patent Application Publication
(10) Pub. No.: US 2025/0090050 A1
(43) Pub. Date: Mar. 20, 2025

(54) METABOLIC SENSOR DEVICE

(71) Applicants: Stoyan Radkov, Gerrards Cross (GB); CENCE INC., Novato, CA (US)

(72) Inventors: Sunshine Holmberg, Lake Elsinor, CA (US); Maziar Zarrehparvar, Novato, CA (US); Dian Song, Irvine, CA (US)

(73) Assignee: CENCE INC., Novato, CA (US)

(21) Appl. No.: 18/556,793

(22) PCT Filed: May 10, 2022
(86) PCT No.: PCT/GB2022/000052

§ 371 Date: Oct. 23, 2023

(30) Foreign Application Priority Data
May 11, 2021 (GB) GB2106717.8
Dec. 27, 2021 (GB) GB2119047.5

(51) Int. Cl. A61B 5/145; A61B 5/1477; G01N 27/30; G01N 27/327

(52) U.S. Cl. CPC A61B 5/14517; A61B 5/1477; G01N 27/308; G01N 27/3278; A61B 2503/10

(57) ABSTRACT

The present invention relates to a device for sensing a metabolite such as chemicals and biomarkers, in bodily fluids such as sweat. The device comprises a carbon-based electroconductive material. The device can be configured as part of a wearable.

The present invention relates to a device. Specifically, the invention relates to a metabolite such as chemicals and biomarkers sensing device. The present invention relates to a device which comprises a carbon-based material capable of detecting metabolites such as chemicals and biomarkers, in bodily fluids such as for example in sweat. The present invention relates to a device which comprises a carbon-based electroconductive material capable of detecting metabolites such as chemicals and biomarkers, in bodily fluids such as for example in sweat. The present invention relates to a sweat metabolite such as chemicals and biomarkers sensing device, as well as uses, screening assays and a method. The device of the present invention can be configured as part of a wearable.

### BACKGROUND

The current method of monitoring human metabolic activities has been limited to laboratory and medical settings. Metabolic activities are heavily reflected in sweat, which has not been accessible through consumer wearables in the past. Currently, most metabolite biomarkers, such as xanthine and uric acid, can only be sampled through invasive methods such as blood draw, fingerstick, and microneedles. In addition, a lack of convenient, continuous-measurement, methods of monitoring biomarkers also limits the usefulness of laboratory-based biomarker information.

There is a perpetual desire for people to understand their bodies better. A major obstacle to this understanding is the inability of most technologies to capture important physiological processes while they are happening. There is a paradigm shift on the way, known as the quantified-self movement, which is nurtured by digitalization of a growing number of behavioral and physiological attributes in individuals and populations.

Traditionally athletes utilize several methods to guide their training. Three of these are: laboratory assays, intuition, and wearables. Laboratory assays can provide a large range of biomarker information but require specialized equipment and trained administrators which is expensive and interferes with training. Results from these assays are often not available in real-time. Laboratory assays usually require blood draws before and after workout. Since data cannot be collected continuously during exercise, opportunities for real-time feedback are limited.

Intuition based methods are subjective and often based on perception of exertion. This method can be misleading and often the sensations that drive the intuition take place after thresholds have been reached, when it is too late to adjust intensity.

Wearable technology hopes to bridge the gap between intuitive and objective data collections. Wearables can be scientific, like assays, and can provide real-time feedback. However, wearable technology is currently limited to a small number of metrics produced from sensors such as EKG, PPG, blood oximetry, skin conductance, etc. and cannot answer all the questions a laboratory blood draw could.

Google, Apple, Garmin, Fitbit, Whoop, Underarmour, Nike, Gatorade and other wearable technology companies do not, currently, have access to a technology with metabolite sensing capabilities.

There is an unmet need to monitor health, fitness and wellness accurately, actionably, and comprehensively. This unmet need is at least in part addressed by the present invention.

### SUMMARY

The present invention relates to a device.

The present invention relates to a device for detecting metabolites, the device comprising a carbon-based sensor. The present invention relates to a device wherein the carbon-based sensor comprises a carbon-based electroconductive material.

More in particular the invention relates to a sweat metabolite such as chemicals and biomarkers sensing device.

The present invention relates to a device which comprises a carbon-based material capable of detecting metabolites such as chemicals and biomarkers in bodily fluids such as sweat.

The present invention relates to a device which comprises a carbon-based electroconductive material capable of detecting metabolites such as chemicals and biomarkers, in bodily fluids such as sweat.

Here is provided a carbon-based wearable sweat sensor device that analyzes sweat metabolites such as chemicals and biomarkers continuously, in real-time. The present device can be used to benefit athletic performance, fitness, and general health.

According to one aspect, there is provided a metabolite sensing device.

In some embodiments the device comprises a carbon-based electroconductive material.

In some embodiments the device can detect metabolites such as chemicals and biomarkers.

In some embodiments the device comprises a carbon-based material capable of detecting metabolites such as chemicals and biomarkers, in bodily fluids.

In some embodiments the device analyzes metabolites such as chemicals and biomarkers continuously, in real-time.

In some embodiments, the carbon-based material is selected from the group comprising carbon nanofibers (CNFs), carbon nanotubes (CNTs), Stress Activated Pyrolytic Carbon (SAPC) or mixtures thereof.

In some embodiments the carbon-based material comprises SAPC/CNT hybrid or composite.

In some embodiments the carbon-based material comprises SAPC.

In some embodiments the device comprises a carbon-based material capable of detecting metabolites such as chemicals and biomarkers, in bodily fluids such as sweat.

In some embodiments the device analyzes sweat metabolites such as chemicals and biomarkers continuously, in real-time.

According to a further aspect, there is provides a device capable of detecting sweat metabolites such as chemicals and biomarkers continuously, in real-time.

According to a further aspect, there is provides a device capable of detecting sweat metabolites such as chemicals and biomarkers continuously, in real-time for use in athletic performance, fitness, and general health.

According to a further aspect, there is provided a method for detecting sweat metabolites such as chemicals and biomarkers continuously, in real-time comprising the steps of providing a device, contacting the device with a bodily fluid and determining the presence of a metabolite, wherein the device comprises a carbon-based material.

According to a further aspect, there is provided an assay for detecting metabolites using a device as described here, wherein an alteration in the levels of a metabolite in a bodily fluid is an indication of abnormal metabolic activity.

According to a further aspect, there is provided a carbon-based wearable sweat sensor device. In some embodiment, the wearable sweat sensor device comprising a carbon-based electroconductive material.

According to some embodiments, there is provided a device for detecting a metabolite selected from the group consisting of xanthine, hypoxanthine, urea, uric acid, ascorbic acid, dopamine, serotonin, norepinephrine, iodine, epinephrine, oxygen, L-Tyrosine, L-Methionine, hydrogen peroxide, glucose, purines and lactate.

In some preferred embodiments, there is provided a device for detecting the metabolite xanthine. In some preferred embodiments, there is provided a device for detecting the metabolite hypoxanthine. In some preferred embodiments, there is provided a device for detecting the metabolite urea. In some preferred embodiments, there is provided a device for detecting the metabolite uric acid. In some preferred embodiments, there is provided a device for detecting purines. In some preferred embodiments, there is provided a device for detecting the metabolite ascorbic acid. In some preferred embodiments, there is provided a device for detecting the metabolite dopamine. In some preferred embodiments, there is provided a device for detecting the metabolite serotonin. In some preferred embodiments, there is provided a device for detecting the metabolite norepinephrine. In some preferred embodiments, there is provided a device for detecting glucose.

### BRIEF DESCRIPTION OF THE DRAWINGS

FIG. 1. Direct detection of different metabolites in sweat

FIG. 1.A. Direct detection of Uric acid, Xanthine and hypoxanthine in sweat

FIG. 1.B. Xanthine Sensitivity curve

FIG. 1.C. Xanthine differential pulse voltammetry (DPV) curves

FIG. 2 Sweat metabolite sensor device

FIG. 2.A. Sweat metabolite sensor prototype 1.0

FIG. 3. Sweat metabolite sensor devise in use on an athlete

FIG. 4. Graph of in vitro experiments on sweat using device comprising SAPC

FIG. 5. Electrochemical signals signified by deviations from this background often in the form of a Gaussian curve

FIG. 6. Detection of the metabolite Uric Acid

FIG. 7. Detection of Tyrosine

FIG. 8. Detection of Hypoxanthine

FIG. 9. On skin summary data during workout based on FIGS. 10A and 10B-xanthine biomarker or hypoxanthine biomarker

FIG. 10A. Graph representing Aerobic activity collected from running in place and Anaerobic done in sets consisting of 2-3 min of lunges->squats->calf lifts->30 sec break.

FIG. 10B.—Heart rate as measured during FIG. 10A.

### DETAILED DESCRIPTION

Throughout this disclosure, various scientific publications, patents and published patent specifications are referenced by an identifying citation. The disclosures of these publications, patents and published patent specifications are hereby incorporated by reference into the present disclosure to more fully describe the state of the art to which this disclosure pertains.

As used herein, certain terms may have the following defined meanings.

As used in the specification and claims, the singular form “a,” “an” and “the” include singular and plural references unless the context clearly dictates otherwise. For example, the term “metabolite” refers to chemicals, biomarkers, amino acids, peptides, hormones, saccharides such as sugars for example xanthine, hypoxanthine, urea, uric acid, purines, ascorbic acid, dopamine, serotonin, norepinephrine, iodine, epinephrine, oxygen, L-Tyrosine, L-Methionine, hydrogen peroxide, glucose and lactate, ATP and ADP as released or present in bodily fluids such as sweat, blood, plasma or interstitial fluid.

Below in Table 1 there are listed a small selection of example markers in sweat, and their applications in diet, performance athletics, health, and disease.

Sweat is derived from interstitial fluids and provides a rich source of access to many important biomarkers.

| Detectable Biomarkers | | | | | | |
|---|---|---|---|---|---|---|
| | Tested | Tested | | Exercise Intensity | Exercise Recovery | |
| | on-skin | in vitro | Diet | (VO₂ Max) | (Oxidative Stress) | Medical Relevance |
| Xanthine | [symbol] | [symbol] | ✓ | ✓ | ✓ | Gout/ATP Metabolism |
| Uric Acid | [symbol] | [symbol] | ✓ | ✓ | ✓ | Gout/Hypertension |
| | | | | | | Glucose Tolerance |
| Vitamin C | [symbol] | [symbol] | ✓ | ? | ✓ | Vitamin C Deficiency |
| Tyrosine | [symbol] | [symbol] | ✓ | ✓ | ? | Phenylketonuria |
| H₂O₂ | [symbol] | | Mediator for | Mediator for | Mediator for | Various disease markers |
| | | | additional | additional | additional | via enzymatic assays |
| | | | markers | markers | markers | |
| Dopamine | [symbol] | | ? | ? | ? | Mental Health |
| Serotonin | [symbol] | | ? | ? | ? | Mental Health |
| Epinepherine | [symbol] | | ? | ? | ? | Mental Health |

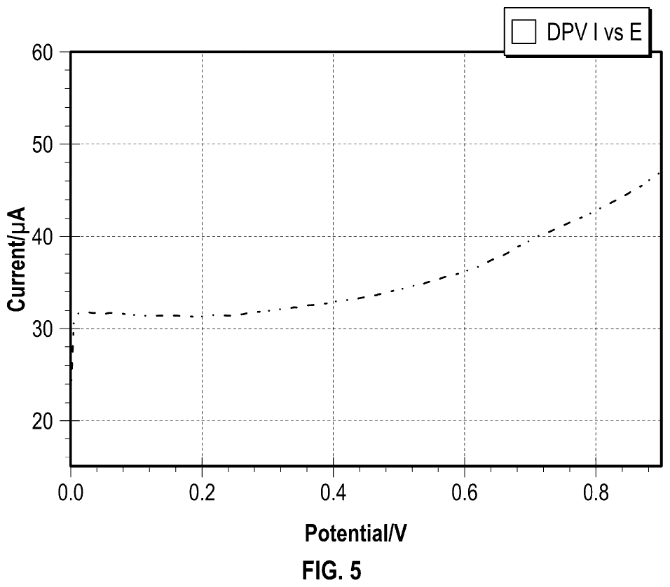

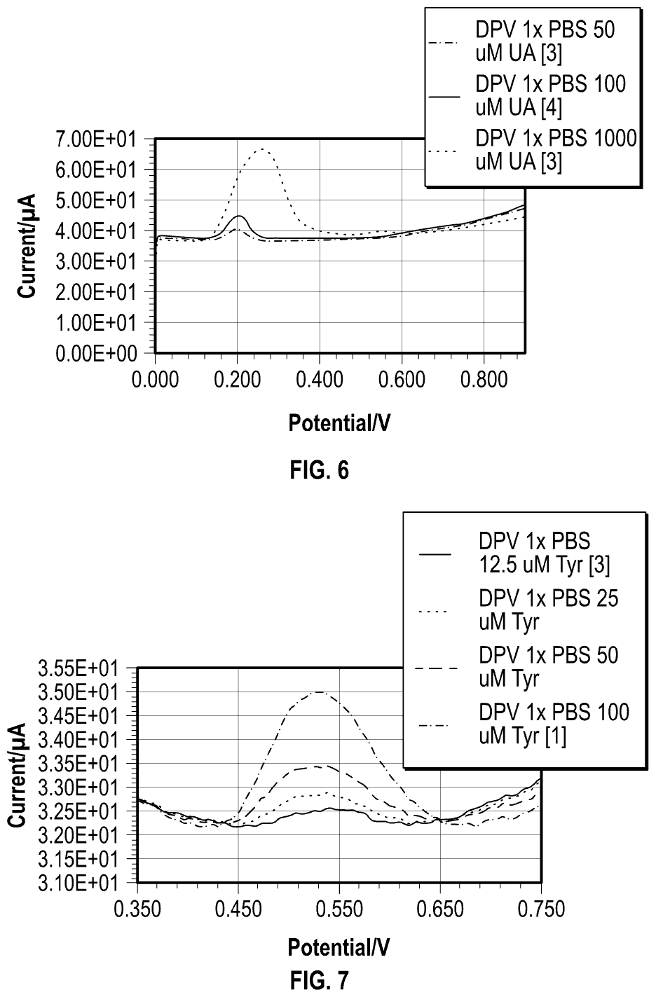

As used herein the term “carbon-based material” refers to a material which base material is carbon such as carbon nanofibers (CNFs), carbon nanotubes (CNTs), SAPC and mixtures thereof or as described in U.S. Pat. No. 7,790,135 (incorporated herein by reference) and WO2020/037334A1 (incorporated herein by reference). The term “Stress Activated Pyrolytic Carbon” or “SAPC,” refers to a composition based on carbon and nitrogen that is the subject of this disclosure. SAPC, by the definition of this disclosure, refers to an inclusion of a semi-graphitic carbon material characterized by wavy graphite planes ranging from 0.1 nm to 1 nm and oriented parallel to an axis of a respective carbon nanofiber. The term “oriented parallel” refers to the general direction of a main surface of a particular graphitic plane and takes into account that variations in angle are expected to occur based on the “wavy” physical nature of SAPC (FIG. 5 and FIG. 6 of WO2020/037334A1).

The term “appreciable” refers to some quality, e.g., a particular amount or percentage, of something that results is a detectable difference either from a defined base-line or over prior-art materials. Such quality should be detectable either directly through observation, e.g., through a Scanning Electron Microscope (SEM) or a Tunneling electron microscope (TEM), or indirectly through physically and/or chemically detectable properties, e.g., electron mobility, conductivity, etc.

All numbers or numerals as used herein that indicate amounts, ratios of materials, physical properties of materials, and/or use are to be understood as modified or qualified by the term “about,” except as otherwise explicitly indicated.

As used herein, the term “about” includes the recited number or number and +/−10% from the recited numeral or number. By way of non-limiting example, the term “about ten (10)” would encompass nine (9) to eleven (11) or 9-11.

As used herein, the term “subject” means any animal, such as a vertebrate, preferably a mammal such as human.

As used herein, the term “treatment” or “treating” refers to an amelioration, prophylaxis, or reversal of a disease or disorder, or of at least one discernible symptom thereof. In some embodiments, “treatment” or “treating” refers to an amelioration, prophylaxis, or reversal of at least one measurable physical parameter related to the disease or disorder being monitored or detected by the device of the present invention, not necessarily discernible in or by the mammal. In some embodiments, “treatment” or “treating” refers to inhibiting or slowing the progression of a disease or disorder, either physically, e.g., stabilisation of a discernible symptom, physiologically, e.g., stabilisation of a physical parameter, or both. In some embodiments, “treatment” leads to partial or complete remission of the disease or disorder.

In one aspect, there is provided a device for detecting metabolites. The device comprises a carbon-based sensor. The carbon-based sensor comprises a carbon-based electroconductive material.

The invention relates to a sweat metabolite such as chemicals and biomarkers sensing device.

In some embodiments, the carbon-based material comprises carbon nanofibers (CNFs), carbon nanotubes (CNTs), SAPC or mixtures thereof.

In some embodiments the carbon-based material is SAPC/CNT hybrid or composite.

In some embodiments the carbon-based material is SAPC.

The present invention relates to a device which comprises a carbon-based electroconductive material capable of detecting metabolites such as chemicals and biomarkers, in bodily fluids such as for example in sweat.

Here is provided a carbon-based wearable sweat sensor device that analyzes sweat metabolites such as chemicals and biomarkers continuously, in real-time. The present device can be used to benefit athletic performance, fitness, and general health. The sensor can be housed in a cassette component in the device. The cassette component can be configured to be replaceable. Sensor must be in contact with the tested bodily fluid.

According to one aspect, there is provided a metabolite sensing device.

In some embodiments the device comprises a carbon-based electroconductive material.

In some embodiments the device can detect metabolites such as chemicals and biomarkers.

In some embodiments the device comprises a carbon-based material capable of detecting metabolites such as chemicals and biomarkers, in bodily fluids.

In some embodiments the device comprises a carbon-based material capable of detecting metabolites such as chemicals and biomarkers, in bodily fluids such as sweat.

In some embodiments the device analyzes sweat metabolites such as chemicals and biomarkers continuously, in real-time.

According to a further aspect, there is provides a device capable of detecting sweat metabolites such as chemicals and biomarkers continuously, in real-time.

According to a further aspect, there is provides a device capable of detecting sweat metabolites such as chemicals and biomarkers continuously, in real-time for use in athletic performance, fitness, and general health.

According to a further aspect, there is provided a method for detecting sweat metabolites such as chemicals and biomarkers continuously, in real-time comprising the steps of providing a device, contacting the device with a bodily fluid and determining the presence of a metabolite, wherein the device comprises a carbon-based material.

According to a further aspect, there is provided a device in an assay to detect metabolites such as chemicals and biomarkers. In some embodiments, there is provided an assay for detecting metabolites using a device as described here, wherein a modification in the levels of a metabolite in a bodily fluid is an indication of abnormal metabolic activity. For example, when compared with the level of the metabolite in one or more subjects or when compared to the level of the metabolite in the same subject observed at an earlier time point (e. g. comparison with a “base line” level) for example before workout.

In some embodiments of the assay, the above base level detection of the metabolites is an indication of an altered metabolic activity. In some embodiments of the assay, the above base level detection of the metabolites is an indication of an altered physiological status.

In some embodiments of the assay, the above base level detection of the metabolites is an indication of a symptom, health, phycological or neurological condition. In some embodiments of the assay, the above base level detection of the metabolites is an indication of tumour or cancer condition. In some embodiments of the assay, the above base level detection of the metabolites is an indication of different stage of development of tumour or cancer condition. Examples of different states of cancer include but not limited to cervical cancer, lung cancer, liver cancer, breast cancer, pancreatic cancer, prostate cancer, brain cancer, bone marrow cancer and cancer of the lymphatic system. In some embodiments of the assay, the above base level detection of the metabolites is an indication of cervical cancer stage 1, cervical cancer stage 2, cervical cancer stage 3, cervical cancer stage 4. In some embodiments of the assay, the above base level detection of the metabolites is an indication of the propensity of the cancer to be metastatic or disseminate to other tissues or organs.

In some embodiments, the above base level detection of the metabolites can track or monitor onset or progression of phycological or neurological conditions. In some embodiments, the above base level detection of the metabolites can track or monitor onset or progression of tumour and/or cancer.

In some embodiments, the above base level detection of the metabolites can track or monitor onset or progression of stress or mental health. In some embodiments, the above base level detection of the metabolites can track or monitor onset or progression of stress or metabolic disorders. Examples of metabolic disorders include metabolic syndrome, pre-diabetes, gout and diabetes.

As used herein, the term “alteration” may be used interchangeably with the terms, “alter” or “modify” such as increase or decrease in the level of a metabolite such as a chemical or a biomarker detected and/or analysed by the device of the present invention. In some embodiments, the alteration is at least 0.01%, 0.05%, 0.1%, 0.15%, 0.2%, 0.4%, 1%, 2%, 2.5%, 5%, 10%, 15%, 20%, 25%, 30%, 35%, 40%, 45%, 50%, 55%, 60%, 65%, 70%, 75%, 80%, 85%, 90%, 95% or 100% or greater compared to control or base level. In some embodiments the alteration may be at least 0.1, 0.25, 0.5, 1, 1.5, 2, 2.5, 3, 3.5, 4, 4.5, 5, 5.5, 6, 6.5, 7, 7.5, 8, 8.5, 9, 9.5, or 10-fold or greater compared to control or base level. In some embodiments the alteration is statistically significant. In some embodiments the effectiveness or the device of the present invention is determined qualitatively. In some embodiments the effectiveness of the device of the present invention is determined quantitatively. In some embodiments the alteration is determined qualitatively. In some embodiments the alteration is determined quantitatively. In some embodiments, alteration is assessed by a qualitative step and/or a quantifying step. In further embodiments, the qualitative step and/or a quantifying step is performed on a sample using the device of the present invention. In further embodiments, the qualitative step and/or a quantifying step is performed continuously. In further embodiments, the qualitative step and/or a quantifying step is performed in real time. In some embodiments, the sample is one of a sweat, plasma sample, blood sample, sputum sample, lavage, synovial fluid, or combinations thereof.

According to a further aspect, there is provided a carbon-based wearable sweat sensor device. In some embodiments, the present invention provides a carbon-based wearable sweat sensor device for analysing sweat metabolites such as chemicals and biomarkers continuously. In some embodiments, the present invention provides a carbon-based wearable sweat sensor device for analysing sweat metabolites such as chemicals and biomarkers, in real-time. In some embodiments the present wearable sweat sensor device is used to benefit athletic performance, fitness, and general health.

The present invention is depicted in the claims below:
1. A device for detecting metabolites, the device comprising a carbon-based sensor.
2. A device according to claim 1, wherein the carbon-based sensor comprises a carbon-based electroconductive material.
3. A device according to anyone of claim 1 or 2, wherein the carbon-based material is capable of detecting metabolites.
4. A device according to anyone of the preceding claims wherein the metabolite is a chemical and biomarker in a bodily fluid.
5. A device according to claim 4, wherein the device detects metabolites such as chemicals and biomarkers continuously.
6. A device according to anyone of claims 1 to 5, wherein the device detects metabolites such as chemicals and biomarkers in real-time.
7. A device according to anyone of the preceding claims, wherein the carbon-based material is selected from the group comprising carbon nanofibers (CNFs), carbon nanotubes (CNTs), Stress Activated Pyrolytic Carbon (SAPC) or mixtures thereof.
8. A device according to claim 7, wherein the carbon-based material comprises SAPC/CNT hybrid or composite.
9. A device according to anyone of claims 1 to 7, wherein the carbon-based material comprises SAPC.
10. A device according to anyone of claims 1 to 9, wherein the device detects metabolites such as chemicals and biomarkers in bodily fluids.
11. A device according to claim 10, wherein the bodily fluid is sweat.
12. A device according to anyone of claims 1 to 11, wherein metabolites are analysed continuously.
13. A device according to anyone of claims 1 to 11, wherein the metabolites are analysed in real-time.
14. A device according to anyone of claims 1 to 13, for use in assisting athletic performance, fitness, and general health.
15. A device according to anyone of claims 1 to 14, for detecting a metabolite selected from the group consisting of xanthine, hypoxanthine, urea, uric acid, ascorbic acid, dopamine, serotonin, norepinephrine, iodine, epinephrine, oxygen, L-Tyrosine, L-Methionine, hydrogen peroxide, purines, glucose and lactate.
16. A device according to claim 15, wherein the metabolite is xanthine.
17. A device according to claim 15, wherein the metabolite is hypoxanthine.
18. A device according to claim 15, wherein the metabolite is urea or purines.
19. A device according to claim 15, wherein the metabolite is uric acid.
20. A device according to claim 15, wherein the metabolite is ascorbic acid.
21. A device according to claim 15, wherein the metabolite is dopamine.
22. A device according to claim 15, wherein the metabolite is serotonin.
23. A device according to claim 15, wherein the metabolite is norepinephrine.
24. A device according to claim 15, wherein the metabolite is glucose.
25. A method for detecting sweat metabolites, the method comprising the steps of:
   a. providing a device,
   b. contacting the device with a bodily fluid, and
   c. determining the presence of a metabolite,
   wherein the device comprises a carbon-based material.
26. A method according to claim 25, wherein the carbon-based material is selected from the group comprising carbon nanofibers (CNFs), carbon nanotubes (CNTs), Stress Activated Pyrolytic Carbon (SAPC) or mixtures thereof.
27. An assay for detecting metabolites using a device according to anyone of claims 1 to 24, wherein an alteration in the levels of a metabolite in a bodily fluid is an indication of abnormal metabolic activity.
28. An assay according to claim 27, wherein the alteration in the levels of a metabolite is relative to base level of the metabolite.
29. An assay according to anyone of claim 27 or 28, wherein above base level detection of the metabolites is an indication of a symptom, health, phycological or neurological condition.
30. An assay according to anyone of claims 27 to 29, wherein the above base level detection of the metabolites is an indication of tumour or cancer condition.
31. A wearable comprising a device according to anyone of claims 1 to 30.

In order that the invention may be readily understood and put into practical effect, particular embodiments will now be described by way of the following non-limiting examples.

### EXAMPLES

### Example 1—Solution and Advantage of the Device Comprising a Carbon-Based Material as the Sensor

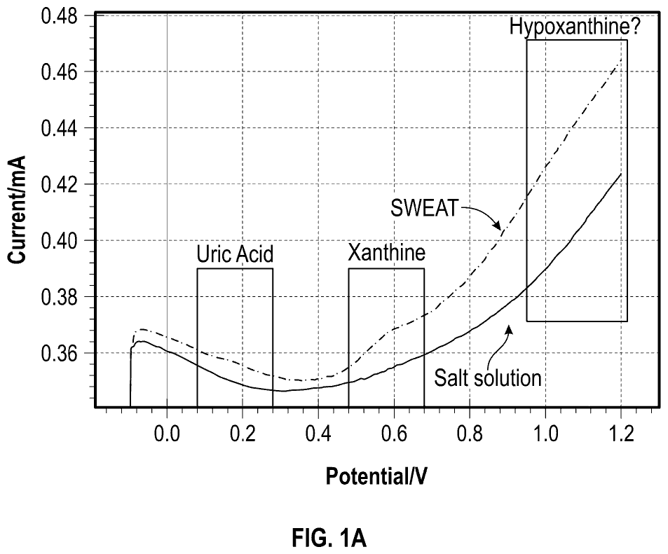

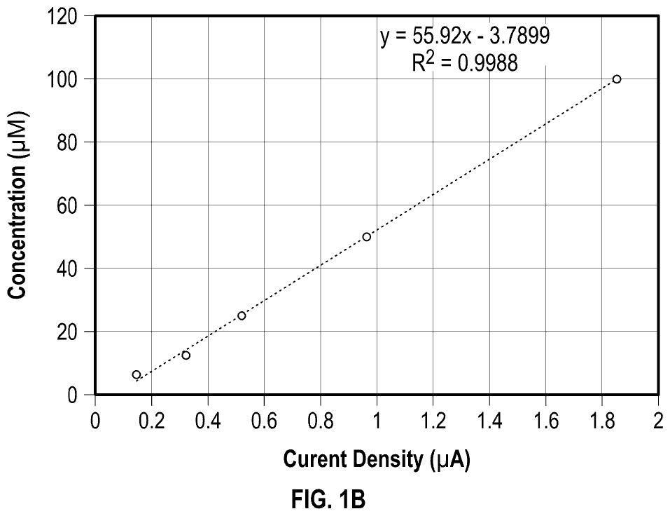

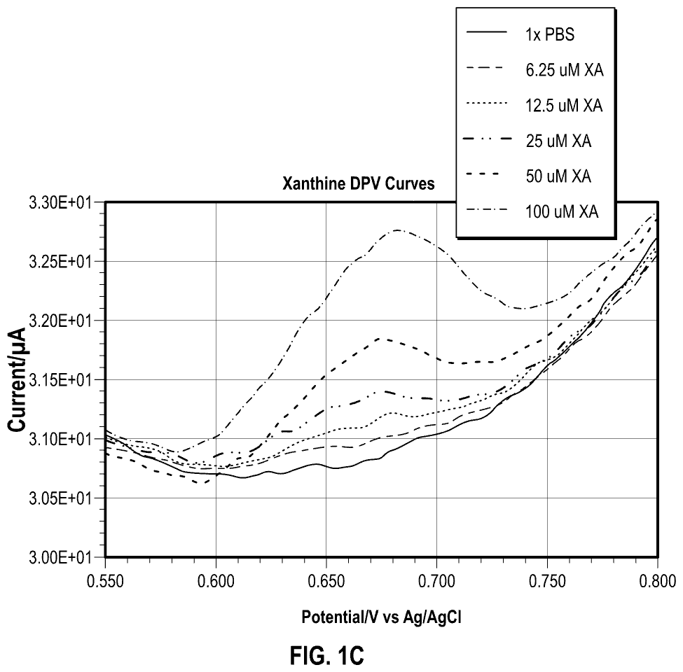

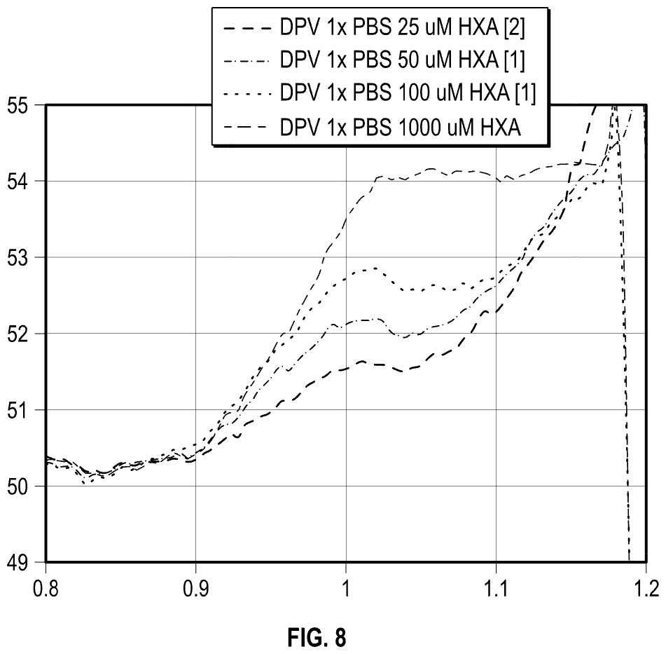

The solution and competitive advantages derive from ability of the device to detect metabolic biomarkers directly, continuously, conveniently, and inexpensively from an individual's bodily fluids such as from sweat. The device can detect uric acid, urea, xanthine and hypoxanthine—FIGS. 1 A, B, and C and FIG. 8. Other metabolites which can be detected with the device include purines.

The unique Peak Potential for xanthine is approximately between 0.65 and 0.7 potential/V (Please see FIG. 1.C. showing different concentrations of xanthine using a differential pulse voltammetry (DPV) curve).

The device also detects uric acid as shown in FIG. 6 with a unique Peak Potential signature at approximately between 0.2 to 0.3 potential/V.

The device is further capable of detecting tyrosine as shown in FIG. 7 with a unique Peak Potential signature at approximately between 0.48 to 0.52 potential/V.

Traditional access to these biomarkers requires invasive sample collection, followed by complicated assays. Our wearable technology, shown on the right below, is more practical and convenient than using invasive means such as needles (shown on the left below). Unique biomarker information collected and collated by our sensor can be displayed in real-time without having to send specimens off to a lab.

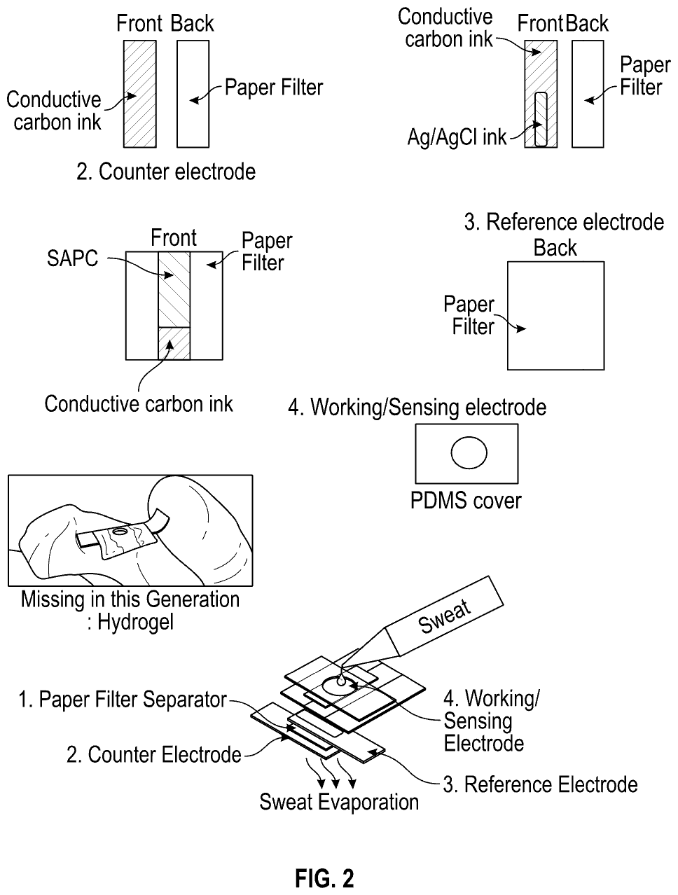

FIG. 2 there us depicted one method of making the device. The device comprises a front and back plate where the front place incorporates the conductive carbon ink or carbon-based electroconductive material which is the sensor of the metabolites and can be made from for example SAPC, CNFs, CNTs or mixtures thereof such as SAPC/CNT.

The device depicted in FIG. 2 comprises an electrode assembly.

The electrode assembly comprises: 1. Paper filter separator; 2. Counter electrode; 3. Reference electrode; and 4. Working/sensing electrode (sensor). The electrode assembly is linked to a potentiostat, not shown, for wire or wireless transmittal of the detected electric signals or unique differential pulse voltammetry (DPV) signatures of the metabolites to a base station for further analysis or instant display. The base station can be an electronic device such as a computer, tablet, phone or printer.

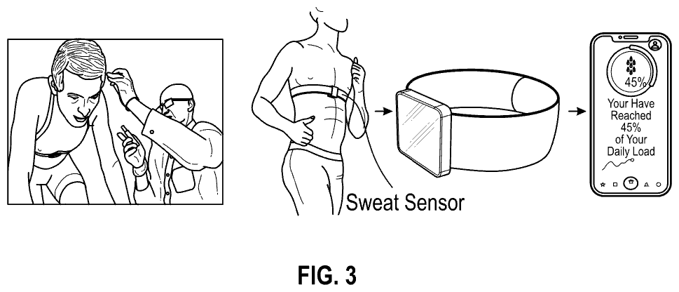

In FIG. 3 there is depicted a sweat metabolite sensor device in use in situ on an athlete during workout.

The device of the present invention can collect data on different metabolites such as chemical or biomarkers during exercise and display these in real-time. The metabolites can be related to muscle exertion, oxidative stress, mental health, and organ function. Elite athletes and their coaches can benefit from the data during exercise to modify and optimise training in real-time. Patients and caregivers can use the data to track health conditions and disease onset or progression. Patients and caregivers can use the data to track onset or progression of phycological or neurological conditions. Patients and caregivers can use the data to track onset or progression of stress. General consumers can use the data to supplement self-tracking.

### Example 2—Graph of In Vitro Experiments on Sweat Fluid Using Device Comprising SAPC

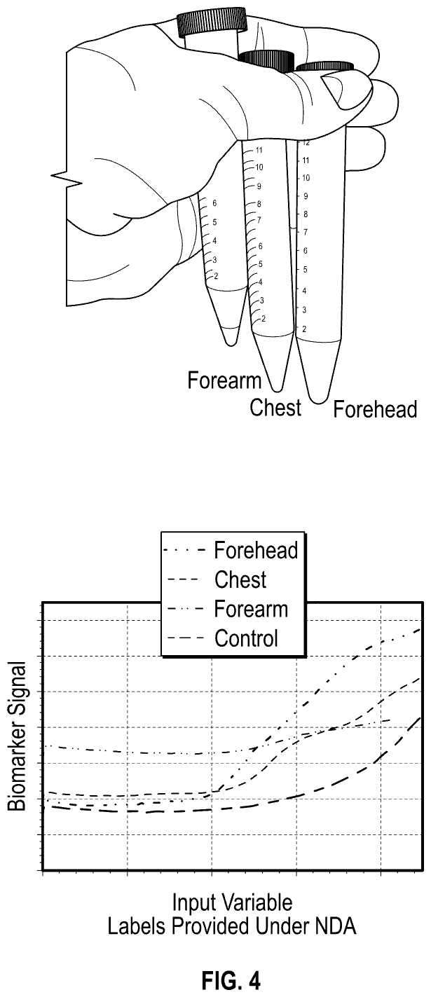

The graph in FIG. 4 shows in vitro results obtained using sweat sampled from the body, which were detected and analyzed using the device described herein. The sensing carbon-material of the device used in this example is SAPC.

The signals varied significantly depending on the body location from which they were sampled. The forehead, the chest, and the forearm each excrete different quantities of important biomarkers. This may have profound consequences for our understanding of local metabolic activity.

In FIG. 5 we have shown electrochemical signals which are signified by deviations from this background often in the form of a Gaussian curve. In electrochemical reaction it is not possible to completely quench background from capacitance and other common reaction like hydrolysis (i.e. background is never perfectly flat).

Electrochemical signals are signified by deviations from this background often in the form of a Gaussian curve. The signature characteristic of electrochemical signal is defined by the peak voltage potential, which is a thermodynamic characteristic of the electrochemical reaction occurring between the electrode (SAPC) and the specific analyte. In order words the analyte measured can be distinguished by the peak potential. Below are examples of known species that can be measured in vitro in a phosphate saline buffer at pH 7.4 (physiological).

### Example 3. Graphs of In Vitro Experiments on Sweat Fluid Using Device Comprising SAPC

The Peak Potential which is detected by the device of our invention, is potentially affected by pH changes. However, we have observed that any pH change is exhibited in a predictable manner. In this connection, we have established that 1 (one) point of change in pH will see about 0.12 mV shift in the positive voltage access.

In particular, human sweat pH ranges from 4 to 7. Average is about 5.5-6. Therefore, we can make accurately calculate and establish the Peak Potential of metabolites in fluids with different pH values upon establishing the exact pH value of the fluid tested by the devise.

### Example 4. Graph Representing Metabolite Detection by Placing the Present Device on a Person Such as on the Skin—In Vivo or In Situ on the Subject

In the present experiment, data is collected in vivo or in situ with the device fitted on the subject.

Each scan of Current/Voltage curve takes ˜35 sec.

The concentration is taken from the peak at around 0.25/0.7/1.3 V for Uric Acid/Xanthine/Hypoxanthine respectively.

Concentration calculated from peak current density and interpreted through sensitivity curve formed from calibrating the sensor prior to in vivo test (linear correlation between current and concentration of metabolite measured).

Data collected from a typical leg day workout.

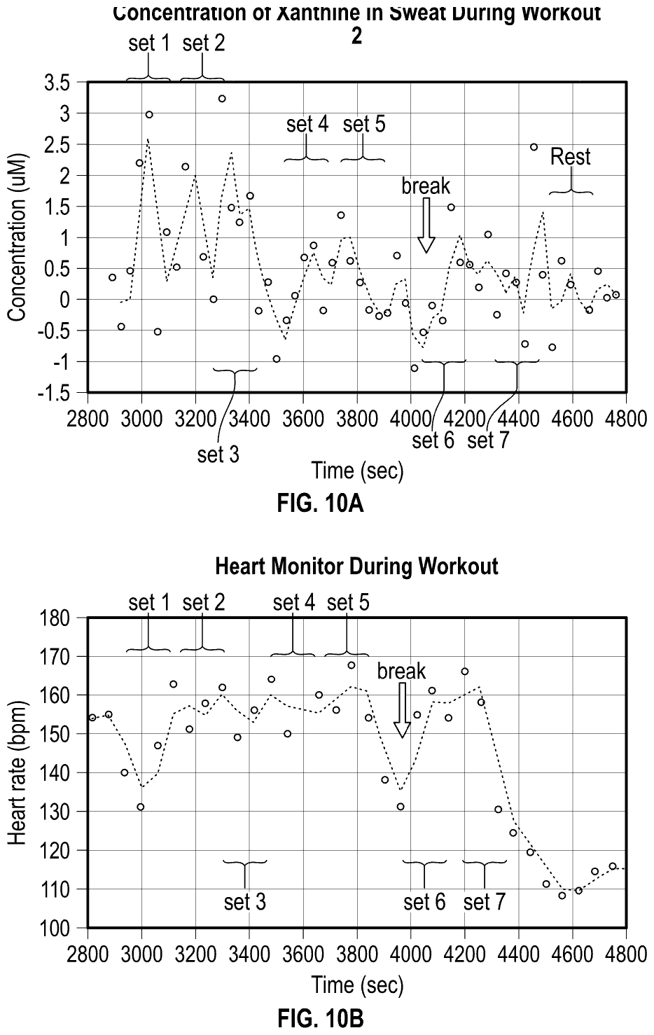

Aerobic collected from running in place-FIG. 10A-concentration of xanthine. Base level of xanthine can be determined per subject or across a group of subjects. The group of subjects can be for example the same age. Other criteria can be used to group subjects and these would be determined on the measured metabolite or health condition under consideration.

In FIG. 10B we summarise the heart rate (a standard physical parameter) determined concurrently with measurements taken during the assessments depicted and in FIG. 10A. The heart rate measurements can be used to track against the data obtained from the device and compare a given subject's levels of tested metabolites which is then correlated against the subject's heart rate. Other standard parameters such as physiological or biochemical, can also be used for correlation against particular metabolite levels as determined by the device of the present invention such as for example, cardiovascular blood pressure, systolic blood pressure, diastolic blood pressure, mean arterial blood pressure, glucose level, ketone levels, purine measurements and others.

Anaerobic done in sets consisting of 2-3 min of lunges->squats->calf lifts->30 sec break-FIGS. 10A and 10B.

Sections marked “break” are breaks taken longer than the standard 30 sec

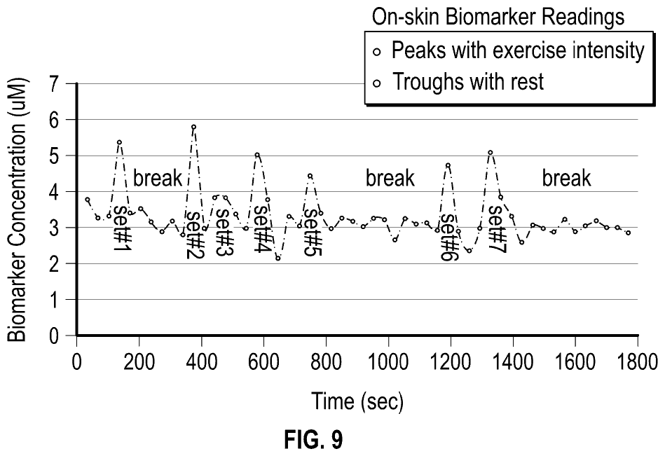

Summary data is plotted as Concentration (μM) vs. Time (sec) as shown in FIG. 9 for xanthene. Similar results were obtained for other metabolites as contemplated by the present invention.

The line is moving average trendline created automatically by Excel.

The disclosure illustratively described herein can suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognised that various modifications are possible within the scope of the disclosure claimed. It will also be appreciated that the method(s), use(s), compositions(s) and/or administrations(s) may be subject to numerous rearrangements, modifications and substitutions without departing from the scope of the present disclosure as set forth and defined by the following claims.

1. A device for detecting metabolites, the device comprising a carbon-based sensor.

2. A device according to claim 1, wherein the carbon-based sensor comprises a carbon-based electroconductive material.

3. A device according to claim 2, wherein the carbon-based material is capable of detecting metabolites.

4. A device according to claim 1, wherein the metabolite is a chemical and biomarker in a bodily fluid.

5. A device according to claim 4, wherein the device detects metabolites such as chemicals and biomarkers continuously.

6. A device according to claim 1, wherein the device detects metabolites such as chemicals and biomarkers in real-time.

7. A device according to claim 2, wherein the carbon-based material is selected from the group comprising carbon nanofibers (CNFs), carbon nanotubes (CNTs), Stress Activated Pyrolytic Carbon (SAPC) or mixtures thereof.

8. A device according to claim 7, wherein the carbon-based material comprises SAPC/CNT hybrid or composite.

9. A device according to claim 7, wherein the carbon-based material comprises SAPC.

10. A device according to claim 1, wherein the device detects metabolites such as chemicals and biomarkers in bodily fluids.

11. A device according to claim 10, wherein the bodily fluid is sweat.

12. A device according to claim 1, wherein metabolites are analysed continuously.

13. A device according to claim 1, wherein the metabolites are analysed in real-time.

14. A device according to claim 1, for use in assisting athletic performance, fitness, and general health.

15. A device according to claim 1, for detecting a metabolite selected from the group consisting of xanthine, hypoxanthine, urea, uric acid, ascorbic acid, dopamine, serotonin, norepinephrine, iodine, epinephrine, oxygen, L-Tyrosine, L-Methionine, hydrogen peroxide, purines, glucose and lactate.

16. A method for detecting sweat metabolites, the method comprising the steps of:
a. providing a device,
b. contacting the device with a bodily fluid, and
c. determining the presence of a metabolite,
wherein the device comprises a carbon-based material.

17. A method according to claim 16, wherein the carbon-based material is selected from the group comprising carbon nanofibers (CNFs), carbon nanotubes (CNTs), Stress Activated Pyrolytic Carbon (SAPC) or mixtures thereof.

18. An assay for detecting metabolites using a device according to claim 1, wherein an alteration in the levels of a metabolite in a bodily fluid is an indication of abnormal metabolic activity.

19. An assay according to claim 18, wherein the alteration in the levels of a metabolite is relative to base level of the metabolite.

20. An assay according to claim 19, wherein above base level detection of the metabolites is an indication of a symptom, health, phycological or neurological condition.

21. An assay according to claim 18, wherein the above base level detection of the metabolites is an indication of tumour or cancer condition.

22. A wearable comprising a device according to claim 1.
